(19) United States
(12) Patent Application Publication
(10) Pub. No.: US 2026/0144634 A1
(43) Pub. Date: May 28, 2026

(54) TRANSCATHETER PROSTHETIC HEART VALVES

(71) Applicant: Edwards Lifesciences Corporation, Irvine, CA (US)

(72) Inventors: Lien-Huong T. Hoang, San Juan Capistrano, CA (US); Sergio Delgado, Irvine, CA (US); Tianwen Zhao, Irvine, CA (US)

(21) Appl. No.: 19/452,587

(22) Filed: Jan. 19, 2026

Related U.S. Application Data
(63) Continuation of application No. PCT/US2024/037060, filed on Jul. 8, 2024.
(60) Provisional application No. 63/514,660, filed on Jul. 20, 2023.

(51) Int. Cl. A61F 2/24

(52) U.S. Cl. CPC A61F 2/2418; A61F 2230/0006

(57) ABSTRACT

Prosthetic heart valves including a radially expandable annular frame and valvular structure disposed within the frame, as well as methods and delivery apparatus associated therewith, are disclosed. The frame can include commissure-supporting cells for attachment of commissures of the valvular structure to the frame. The commissure-supporting cells can be offset from an outflow end of the frame by an offset distance and aligned with gaps in the frame. In some examples, the gaps are defined by a row of discontinuous cells in an outflow portion of the frame. In some examples, the gaps are defined by pairs of cells in an outflow portion of the frame. In some examples, the pairs of cells are enlarged relative to other cell of the frame.

### CROSS-REFERENCE TO RELATED APPLICATIONS

This application is a continuation of PCT Patent Application No. PCT/US2024/037060 filed on Jul. 8, 2024, which application claims the benefit of U.S. Provisional Patent Application No. 63/514,660, filed Jul. 20, 2023, the entire contents of each of these applications being incorporated herein by this reference.

### FIELD

The present disclosure relates to prosthetic heart valves and methods for implanting prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are prosthetic heart valves, delivery apparatus, and methods for implanting prosthetic heart valves. The disclosed prosthetic heart valves, delivery apparatus, and methods can, for example, provide improved coronary access and/or blood hemodynamics when the prosthetic valve is implanted in a native valve. Further, the disclosed prosthetic heart valves, delivery apparatus, and methods can, for example, provide improved recapturability of the prosthetic heart valve and/or radial compression or collapse of the prosthetic heart valve. As such, the devices and methods disclosed herein can, among other things, overcome one or more of the deficiencies of known prosthetic heart valves and their delivery apparatus.

A prosthetic heart valve can comprise a frame and a valvular structure coupled to the frame. In addition to these components, a prosthetic heart valve can further comprise one or more of the components disclosed herein.

In some examples, a prosthetic heart valve can include an outflow row of discontinuous cells at an outflow end of the frame.

In some examples, each of the discontinuous cells is unattached to other cells in the outflow row.

In some examples, the discontinuous cells at least in part define a plurality of gaps in the frame.

In some examples, each of the discontinuous cells extends from a pair of enlarged cells in a row of cells upstream of the outflow row of cells.

In some examples, the enlarged cells are enlarged relative to the discontinuous cells.

In some examples, the enlarged cells are enlarged relative to commissure-supporting cells.

In some examples, each of the plurality of gaps is further defined by at least outflow portions of the enlarged cells.

In some examples, apices of the discontinuous cells form the outflow end of the frame.

In some examples, at least the outflow portions of the discontinuous cells are angled inward toward a longitudinal axis of the frame.

In some examples, at least the outflow portions of the discontinuous cells are angled outward from a longitudinal axis of the frame.

In some examples, an outflow end row of cells comprises pairs of cells.

In some examples, the pairs of cells are pairs of enlarged cells.

In some examples, the pairs of cells are mirrored pairs of cells.

In some examples, the pairs of cells at least in part define a plurality of gaps in the frame.

In some examples, the apices of the pairs of cells can form the outflow end of the frame.

In some examples, at least the outflow portions of cells in the pairs of cells are angled inward toward the longitudinal axis of the frame.

In some examples, each of the gaps is aligned with one of a plurality of commissure-supporting cells in the upstream row of cells.

In some examples, a commissure of the valvular structure is attached to each of the commissure-supporting cells.

In some examples, each of the commissure-supporting cells is offset from the outflow end of the frame by an offset distance.

In some examples, each of the commissures and/or the outflow edges of leaflets of the valvular structure is offset from the outflow end of the frame.

In some examples, at least the outflow portions of each of the commissure-supporting cells is angled inward toward a longitudinal axis of the frame.

In some examples, at least the outflow portion of each of the commissure-supporting cells is disposed at an angle in a range of 5° to 35° relative to the longitudinal axis of the frame.

In some examples, at least the outflow portion of each of the commissure-supporting cells is disposed at an angle in a range of 5° to 25° relative to the longitudinal axis of the frame.

In some examples, at least the outflow portion of each of the commissure-supporting cells can be disposed at a greater angle relative to the longitudinal axis of the frame than the outflow portions of the discontinuous cells.

In some examples, at least the outflow portion of each of the commissure-supporting cells can be disposed at a greater angle relative to the longitudinal axis of the frame than the outflow portions of cells in the pairs of cells.

In some examples, a prosthetic heart valve comprises one or more of the components recited in Examples 1-30 below.

A delivery apparatus for use a prosthetic implant (such as the exemplary prosthetic valves disclosed herein) can comprise a handle and one or more shafts coupled to the handle.

An assembly can comprise a prosthetic heart valve and a delivery apparatus.

In one representative example, a prosthetic heart valve comprises: (i) an annular frame comprising an inflow end and an outflow end arranged along a longitudinal axis of the frame, the frame comprising a first, outflow row of discontinuous cells, wherein the outflow end of the frame is defined by outflow apices of the discontinuous cells in the outflow row, wherein each discontinuous cell in the outflow row is unattached to other discontinuous cells to define a plurality of gaps in the frame between adjacent discontinuous cells within the outflow row; and a second row of cells upstream of the outflow row of cells comprising pairs of enlarged cells, wherein each cell in the outflow row extends from a respective pair of enlarged cells in the second row, wherein the second row further comprises a plurality of commissure-supporting cells disposed circumferentially between respective pairs of enlarged cells, wherein the enlarged cells are larger than the commissure-supporting cells and the discontinuous cells; and (ii) a valvular structure comprising a plurality of leaflets, the plurality of leaflets forming a plurality of commissures that are connected to struts of the commissure-supporting cells.

In another representative example, an annular frame for a prosthetic heart valve comprises: an inflow end and an outflow end arranged along a longitudinal axis of the frame; a first, outflow row of discontinuous cells, wherein the outflow end of the frame is formed by outflow apices of the discontinuous cells in the outflow row, wherein each discontinuous cell in the outflow row is unattached to other discontinuous cells to define a plurality of gaps in the frame between adjacent discontinuous cells within the outflow row; a second row of cells upstream of the outflow row of cells comprising pairs of enlarged cells, wherein each discontinuous cell in the outflow row extends from a respective pair of enlarged cells in the second row, wherein the second row further comprises a plurality of commissure-supporting cells disposed circumferentially between adjacent ones of the pairs of enlarged cells, wherein the enlarged cells are larger than the commissure-supporting cells and the discontinuous cells; wherein apices of the commissure-supporting cells are axially offset from the outflow end of the frame, toward the inflow end of the frame, by an offset distance.

In another representative example, a prosthetic heart valve comprising: (i) an annular frame comprising an inflow end and an outflow end arranged along a longitudinal axis of the frame, the frame comprising a plurality of pairs of cells circumferentially arranged in an outflow end portion of the frame, wherein a plurality gaps in the frame are defined at least in part by outflow portions of the pairs of cells, and wherein each of the gaps is disposed between respective adjacent ones of the pairs of cells; and a plurality of commissure-supporting cells, each of which is disposed circumferentially between a first pair of cells in the plurality of pairs of cells and an adjacent second pair of cells in the plurality of pairs of cells, wherein each of the commissure-supporting cells is aligned with one of the gaps in the frame and is axially offset from the outflow end of the frame toward the inflow end of the frame; and (ii) a valvular structure comprising a plurality of leaflets, the plurality of leaflets forming a plurality of commissures that are connected to struts of the commissure-supporting cells.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of one example of a prosthetic valve including a frame and a plurality of leaflets attached to the frame.

FIG. 2 is a top perspective view of the exemplary prosthetic valve of FIG. 1.

FIG. 3 is a top perspective view of an exemplary frame for the prosthetic valve of FIGS. 1 and 2.

FIG. 4 is a front view of a front portion of the frame shown in FIG. 3.

FIG. 5 is a perspective view of the leaflet structure of the prosthetic valve of FIGS. 1 and 2 shown prior to being secured to the support frame.

FIG. 6 is a cross-sectional view of the valve of FIGS. 1 and 2.

FIG. 7A is a cross-section of a heart illustrating the prosthetic valve of FIGS. 1 and 2 implanted within the aortic annulus, showing the valvular structure removed and only the front portion of the frame for clarity.

FIG. 7B is a side view of an exemplary delivery apparatus for a prosthetic valve.

FIG. 7C is a side view of a prosthetic heart valve being deployed from a capsule of the exemplary delivery apparatus FIG. 7B.

FIG. 8 is a front view of a front portion of another exemplary frame that can be utilized in a prosthetic valve.

FIG. 9 is a top plan view of the frame of FIG. 8.

FIG. 10 is a perspective view of another example of a prosthetic valve including a frame and a plurality of leaflets attached to the frame.

FIG. 11 is a front view of a front portion of an exemplary frame for the prosthetic valve of FIG. 10.

FIG. 12 is a perspective view of another example of a prosthetic valve including a frame and a plurality of leaflets attached to the frame.

FIG. 13 is a top perspective view of an exemplary frame for the prosthetic valve of FIG. 12.

FIG. 14 is a perspective view of another example of a prosthetic valve including a frame and a plurality of leaflets attached to the frame.

FIG. 15 is a top perspective view of an exemplary frame for the prosthetic valve of FIG. 14.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic heart valves can be utilized to treat various valvular diseases by radially expanding a prosthetic heart valve from a compressed state to its functional size at an implantation site within a diseased native heart valve. However, known valves can have various issues associated therewith. For example, some known prosthetic valves can block or limit coronary access when implanted in a native valve. In another example, when implanted, overhanging tissue (for example, overhanging native leaflet tissue) can extend over or around some known prosthetic valves and can result in insufficient blood flow through the valve, which can lead to thrombosis and/or stroke. In another example, after implantation, some known prosthetic valves can be difficult to recapture during a repositioning procedure and/or a replacement procedure. In yet another example, when implanted in a tricuspid valve, some known prosthetic valves may contact the atrioventricular node (AV node), which can disrupt or affect control of electrical impulses from the atria to the ventricles.

The prosthetic heart valves and associated methods disclosed herein can address one or more of the foregoing issues.

The prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

As illustrated in FIGS. 1-7B, in some examples, a prosthetic valve in accordance with the present disclosure can include an annular frame having an inflow end and an outflow end and a valvular structure disposed in an interior of the frame. The outflow end can be formed by a (first) row cells including a plurality of discontinuous cells, which are unattached to other cells in the outflow end row of cells. In some examples, each of the discontinuous cells extends from a pair of enlarged, mirrored cells, which are included a (second) row of cells upstream of the outflow end row of cells. The discontinuous cells can define a plurality gaps within the frame. The second row of cells can further include commissure-supporting cells disposed circumferentially within the frame between respective pairs of enlarged cells. Each of the commissure-supporting cells can be aligned with one of the gaps in the frame, and the commissure-supporting cells can be axially offset from the outflow end of the frame by an offset distance. In some examples, the offset distance is in a range of 3 mm to 15 mm. In some examples, at least the outflow portion of each of the commissure-supporting cells can be disposed at an oblique angle relative to the longitudinal axis of the frame, and can be angled inward toward the longitudinal axis of the frame. In some examples, each of discontinuous cells in the outflow end row can be angled inward toward the longitudinal axis of the frame. In some examples, a distance between the inflow end of the frame and an outflow portion of the commissure-supporting cells (corresponding, in some examples, to a height of the valvular structure) is a range of 10 mm to 20 mm and a height of the frame from the inflow end to the outflow end is in a range of 15 mm to 32 mm.

The foregoing exemplary prosthetic valve can address one or more of the foregoing issues with various known prosthetic valves and/or can have advantages over various known prosthetic valves. For example, the gaps in the frame and the enlarged cells can enable improved coronary access when the prosthetic valve is implanted in a native valve. In another example, the location of the commissure-supporting cells enables the prosthetic valve to have relatively shorter height of the frame supporting the valvular structure, which can also improve coronary access when the prosthetic valve is implanted in a native valve. In yet another example, the relatively shorter height of the frame can avoid or limit contact with the AV node when the prosthetic valve is implanted in a native tricuspid valve. In another example, the frame can limit or prevent tissue overhang when implanted in a native valve. In yet another example, the inward angled discontinuous cells and outflow portions of the commissure-supporting cells can enable recapture of the prosthetic valve.

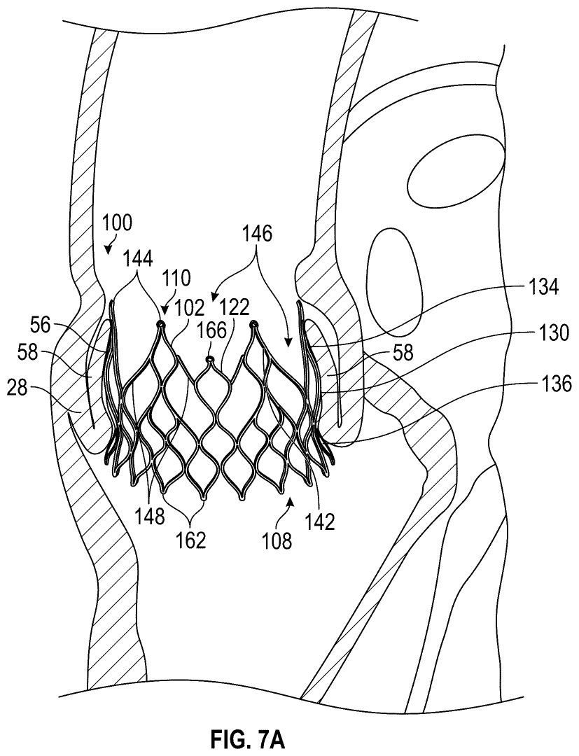

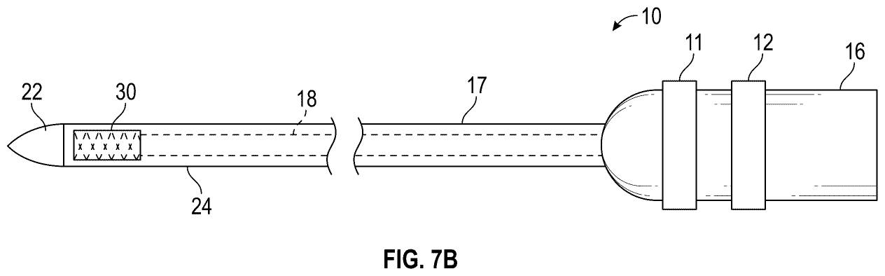

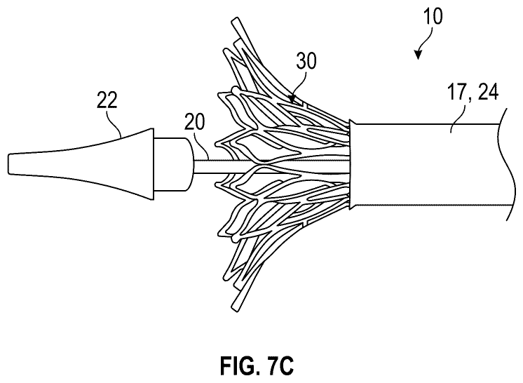

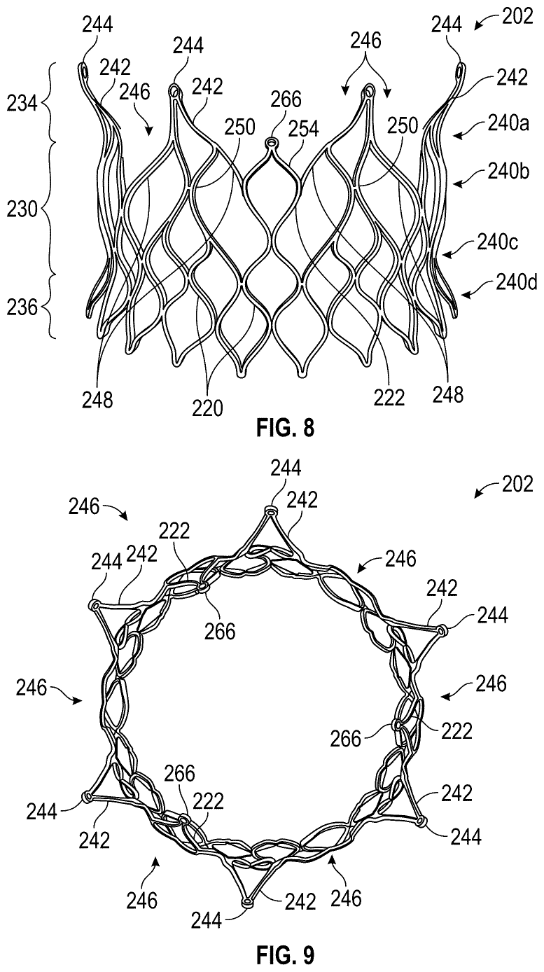

Other exemplary prosthetic valves are illustrated in FIGS. 8-15 and can include one or more of the foregoing features discussed above with respect to the exemplary prosthetic valve of FIGS. 1-7A and/or other features. Further, the exemplary prosthetic valves illustrated in FIGS. 8-15 can provide one or more of the foregoing advantages over known prosthetic valves discussed above with respect to the exemplary prosthetic valve of FIGS. 1-7A and/or other advantages or benefits. An exemplary delivery apparatus that can be utilized with the prosthetic valves disclosed here in shown in FIG. 7B.

### Examples of the Disclosed Technology

Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

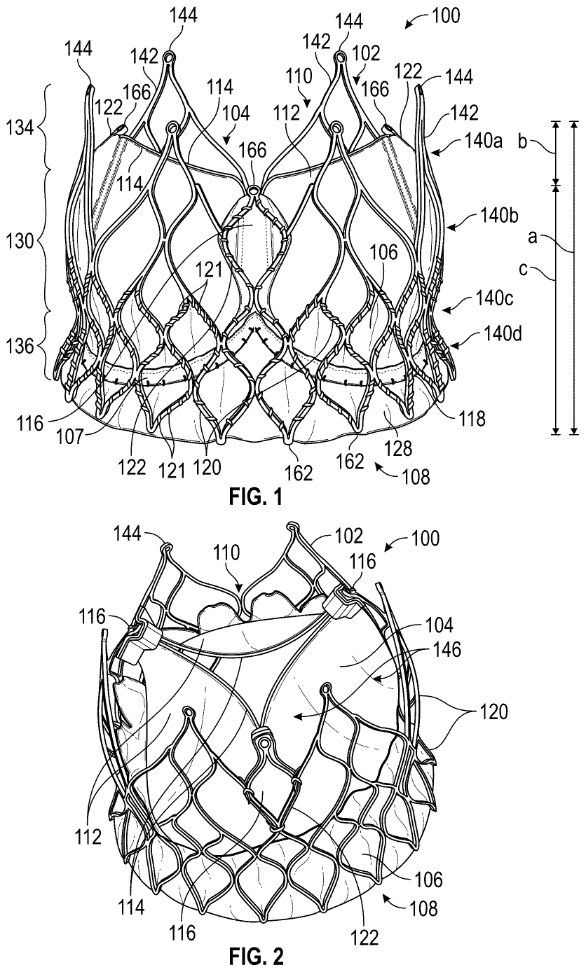

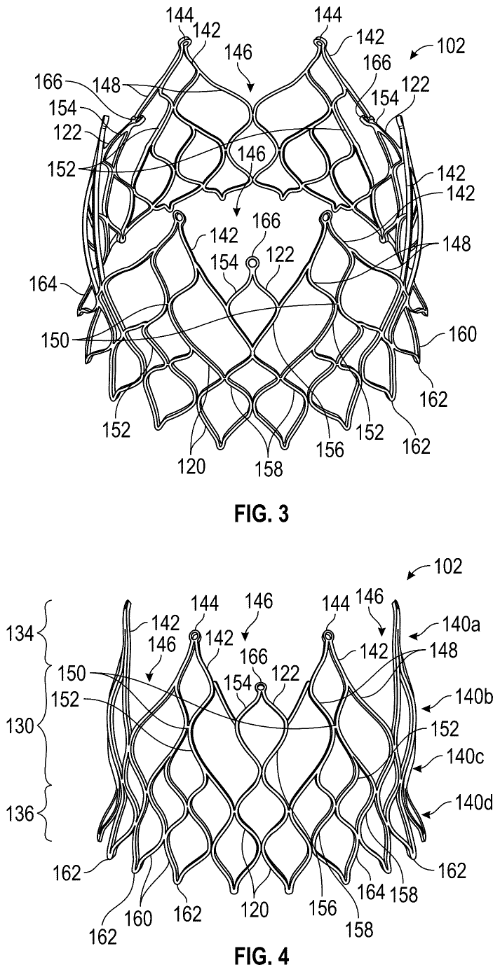

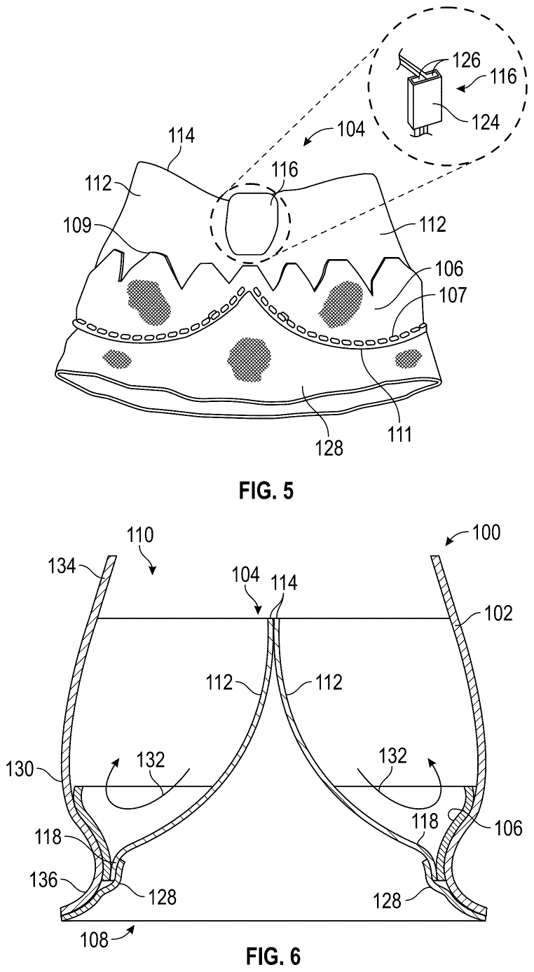

FIGS. 1-7A show an exemplary prosthetic heart valve 100 including an annular frame 102. FIGS. 3-4 and 7A show the frame 102 by itself, while FIGS. 1-2 show the frame 102 with a valvular structure 104 (which can also be referred to as a “valve structure”) mounted within and to the frame 102. FIGS. 5-6 illustrate an exemplary valvular structure 104. The valvular structure 104 can be in the form of a leaflet assembly comprising a plurality of leaflets. FIGS. 1-2 and 5-6 additionally show an optional skirt assembly comprising an inner skirt 106. In some examples, the prosthetic valve can additionally include an outer skirt assembly.

The prosthetic valve 100 can be radially compressible to a compressed state for delivery through the body to a deployment site (for example, a native valve) and expandable to its functional size (shown in FIGS. 1 and 2) at the deployment site. For example, although only the frame 102 is shown in FIG. 7A for simplicity, the figure illustrates the prosthetic valve 100 deployed in an aortic valve. In some examples, the prosthetic valve 100 is self-expanding (for example, fully self-expandable or partially self-expandable); that is, the prosthetic valve can radially expand to its functional size when advanced from the distal end of a delivery sheath. Exemplary delivery apparatus for a self-expanding valve that can be utilized with one or more of the prosthetic valves disclosed herein are described in U.S. Provisional Patent Application No. 63/320,106 and U.S. Pat. Nos. 8,652,202; 9,155,619; and 9,867,700 which are incorporated by reference herein. In some examples, the prosthetic valve can be a balloon-expandable valve that can be adapted to be mounted in a compressed state on a balloon of a delivery catheter. The prosthetic valve can be expanded to its functional size at a deployment site by inflating the balloon. Exemplary delivery apparatus for a balloon-expandable valve that can be utilized with one or more of the prosthetic valves disclosed herein are described in U.S. Provisional Patent Application No. 63/374,343 and U.S. Publication No. 2013/0030519, which are incorporated by reference herein. An exemplary delivery apparatus 10 is shown in FIG. 7B and discussed below.

Returning to FIGS. 1-2, as illustrated therein, the valvular structure 104 can be coupled to and supported inside the frame 102 of the prosthetic valve 100. The valvular structure 104 can be configured to regulate the flow of blood through the prosthetic valve 100, from an inflow end 108 toward an outflow end 110 of the prosthetic valve 100.

The valvular structure 104 can include, for example, a leaflet assembly comprising one or more leaflets 112 made of flexible material. The leaflets 112 can be made from in whole or part, biological material, bio-compatible synthetic materials, or other such materials, such as those described in U.S. Pat. No. 6,730,118, which is incorporated herein by reference. For example, suitable biological material can include bovine pericardium (or pericardium from other sources). The leaflets 112 can extend from the inflow end 108 of the valve 100 toward the outflow end 110. As discussed further below, outflow edge portions 114 of the leaflets 112 and commissures 116 formed between the leaflets 112 can be offset from the outflow end portion 110 of the prosthetic valve 100.

Each leaflet 112 can have an inflow edge portion 118 (which can also be referred to as “cusp edge portion”) opposing the outflow edge portion 114. The inflow edge portions 118 of the leaflets 112 can define an undulating, curved scallop-shaped edge.

The leaflets 112 can be secured to one another at their adjacent sides at the outflow end portions 114 to form the commissures 116. Each of the commissures 116 can be formed at and/or can be secured to a respective commissure support structure 122 (also referred to as “commissure supports” or “commissure-supporting cells”) and/or to other portions of the frame 102 (for example, one or more of the struts 120 of the frame 102 that are adjacent to the commissure-supporting cells 122). In some examples, as shown in FIGS. 1 and 2, the outflow end portion of the leaflet assembly 104 can be secured to the frame 112 at three angularly spaced commissures 116 formed at adjacent side edges of the outflow edge portions 114 of three leaflets 112 in a tricuspid arrangement.

As best shown in FIG. 5, each commissure 116 can be formed by wrapping a reinforcing member 124 around adjacent upper side edge portions 126 (also referred to as “commissure tabs” of the leaflets) of the outflow edge portions 114 of two of the leaflets 112. In some examples, the reinforcing member 124 can be secured to the side edge portions 126 with sutures. In some examples, the sandwiched layers of the reinforcing member 124 and side edge portions 126 of the leaflets 112 can be secured to the struts 120 of the commissure-supporting cells 122 of the frame 102 with sutures. The reinforcing members 124 can reinforce the attachment of the leaflets 112 to the frame 102 so as to minimize stress concentrations at the suture lines and avoid “needle holes” or other wear on the portions of the leaflets that flex during use. The reinforcing members 124 can be made of a suitable fabric (for example, a PET fabric) or natural tissue.

In some examples, the leaflet assembly 104 can include a skirt assembly comprising an upper skirt 106 which can be an annular reinforcing skirt that is secured to the outer surfaces of the inflow edge portions 118 of the leaflets 112 along a scalloped suture line 107 adjacent the inflow end 108 of the prosthetic valve 100. The skirt assembly can also include a lower skirt 128 that is secured to the inner surfaces of the inflow end portions 118 of the leaflets 112. The inflow end portion of the leaflet assembly 104 can be secured to the frame 102 by suturing the skirts 106, 128 to struts 120 of the frame 102 with stitches 121.

An outflow edge 109 of the upper skirt 106 can have a plurality of projections that are shaped to correspond to an adjacent row of struts 120. An inflow edge 111 of the upper skirt 106 can have an undulating, scalloped shape that corresponds to the inflow edges of the leaflets 112. The skirts 106, 128 can function as a sealing member to prevent or decrease perivalvular leakage, to anchor the leaflets 112 to the frame 102, and/or to protect the leaflets 112 against damage caused by contact with the frame 102 during crimping and during working cycles of the prosthetic valve 100. The skirts 106, 128 can be referred to as inner skirts since they disposed inside of the frame 102.

In some examples, the prosthetic valve 100 can further include an outer skirt (not shown) that may be mounted around an outer surface of the frame 102. The outer skirt can function as a sealing member for the prosthetic valve 100 by sealing against the tissue of the native valve annulus and helping to reduce paravalvular leakage past the prosthetic valve 100.

The inner and outer skirts and the reinforcing member 124 can be formed from any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valve can be found, for example, in U.S. Patent Publication No. 2020/0352711, which is incorporated herein by reference.

For example, one or more of the skirts and/or the reinforcing member can be wholly or partly formed of any suitable biological material, synthetic material (for example, any of various polymers), or combinations thereof. In some examples, one or more of the skirts and/or the reinforcing member can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, one or more of the skirts and/or the reinforcing member can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, one or more of the skirts and/or the reinforcing member can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, one or more of the skirts and/or the reinforcing member can comprise a sponge material or foam, such as polyurethane foam. In some examples, one or more of the skirts and/or the reinforcing member can comprise natural tissue, such as pericardium (for example, bovine pericardium, porcine pericardium, equine pericardium, or pericardium from other sources).

Further details regarding the assembly of the leaflet assembly and the assembly of the leaflets and the skirts to the frame can be found, for example, in PCT Application Publication No. WO/2022/261419, filed Oct. 6, 2022, PCT Application Publication No. WO/2023/003696, filed Jul. 7, 2022, and U.S. Pat. No. 10,098,734, which are incorporated herein by reference.

FIG. 6 shows a cross-sectional view of the prosthetic valve 100 illustrating exemplary operation of the valvular structure 104. During diastole, the leaflets 112 collapse to effectively close the valve 100. As schematically shown, the curved or bulging shape of the intermediate section or portion 130 of the frame 102 defines a space between the intermediate section and the leaflets that mimics the Valsalva sinuses. Thus, when the leaflets close, backflow entering the “sinuses” creates a turbulent flow of blood along the upper surfaces of the leaflets, as indicated by arrows 132. This turbulence assists in washing the leaflets 112 and the skirt 106 to minimize clot formation.

As shown in FIGS. 1-2 and discussed above, the radially expandable annular frame 102 supports the valvular structure 104. The frame 102 is illustrated without the valvular structure in FIGS. 3-4 and 7. While only one side of the frame 102 is depicted in FIGS. 4 and 7, it should be appreciated that the frame 102 forms an annular structure having an opposite side that is substantially identical to the portion shown in FIGS. 4 and 7.

The frame 102 can be made of any of various suitable plastically-expandable materials (for example, stainless steel, etc.) or self-expanding materials (for example, Nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. As discussed above, once inside the body, the valve can be advanced from the delivery sheath, which allows the valve to expand to its functional size.

Suitable plastically-expandable materials that can be used to form the frames disclosed herein (for example, the frame 102 and other frames disclosed herein) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 112 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As best illustrated in FIGS. 1-4, the frame 102 includes an outflow end portion 134 and an inflow end portion 136 on opposing ends of the intermediate portion 130. In some examples, the intermediate portion 130 has an overall bulging or outward curved shape that tapers towards each of the outflow end portion 134 and the inflow end portion 136. In some examples, the inflow end portion 136 can flare outward relative to a longitudinal axis of the prosthetic valve 100. In some examples, the outflow end portion 134 can be angled inward toward the longitudinal axis of the prosthetic valve 100.

In some examples, the outflow end portion 134 can include a (first) row of cells 140a, the intermediate portion 130 can include (second and third) rows of cells 140b and 140c, and the inflow end portion 136 can include a (fourth) row of cells 140d. The rows of cells 140a, 140b, 140c, 140d can be formed by interconnected ones of the struts 120.

In some examples, the row of cells 140a can be a first, outflow row including a plurality of discontinuous cells 142. In some examples, the outflow end of the frame 102 is defined by outflow apices 144 of the discontinuous cells 142 in the outflow row 140a. In some examples, each cell 142 in the outflow row 140a can have a generally diamond-shaped or a curved diamond-shaped configuration that is unattached to other cells 142 of the outflow row. As the cells 142 are unattached to each other, the outflow row of cells 140a can (at least in part) define a plurality of gaps 146 in the frame 102 between adjacent ones of the discontinuous cells 142 within the outflow row. In some examples, the gaps 146 are further defined in part by one or more cells in the row 140b (for example, by outflow portions of the enlarged cells 148 in the row 140b). In some examples, the discontinuous cells 142 can be angled inward toward the longitudinal axis of the frame 102. For example, the discontinuous cells 142 can be angled inward at an oblique angle in a range of 1° to 15° relative to the longitudinal axis of the frame. In some examples, each of the outflow apices 144 can include an aperture or other structural feature (for example, a tab) configured for attachment or coupling of the prosthetic valve 100 to a delivery apparatus. In other examples, the discontinuous cells 142 can be parallel relative to the longitudinal axis of the frame 102, or can be angled outward (see, for example, the exemplary frame 202 shown in FIGS. 8 and 9 and discussed below).

As can be seen in FIGS. 1-4, the row of cells 140b is upstream of the row of cells 140a and can include the commissure-supporting cells 122, enlarged cells 148 arranged in mirrored pairs 150, and cells 152 disposed between the enlarged cells 148 in each of the pairs 150. In some examples, the commissure-supporting cells 122 have a generally diamond-shaped or a curved diamond-shaped configuration and can be disposed circumferentially between respective pairs 150 of the enlarged cells 148. In some examples, at least the outflow portions 154 of the commissure-supporting cells 122 can be downstream of the cells 152. In other words, although the commissure-supporting cells 122 are in a same row as the cells 152, they can be offset (in a downstream direction) relative to the cells 152. In some examples, apices 166 of the outflow portions 154 can include an aperture or other structural feature (for example, a tab) configured for attachment or coupling of the prosthetic valve 100 to a delivery apparatus.

In some examples, the enlarged cells 148 can be larger than the cells 122, 152. In some examples, the enlarged cells 148 can be larger than all other cells in the frame 102. In some examples, each of the enlarged cells 148 can have a shape generally corresponding to two substantially diamond-shaped or curved diamond-shaped cells having a central strut removed therebetween. In such examples, each of the enlarged cells 148 can be approximately two times the size of other cells in the frame 102 (for example, each of the enlarged cells 148 can be approximately two times the size of one of the cells 152). In some examples, of the enlarged cells 148 can be in a range of 1.1 to 5, such as a range of 1.5 to 4, 1.8 to 3, 2 to 2.5, etc., times larger than other cells in the frame 102.

In some examples, each of the enlarged cells 148 can have an elongated, curved diamond shape (for example, S-shape), where the curved diamond is elongated between an inflow portion 156 of one of discontinuous cells 142 and outflow portions of cells in the of the row 140c. In some examples, the enlarged cells 148 can be joined at a center point of the pair 150, and each enlarged cell 148 can have a mirrored configuration relative to the other enlarged cell 148 in the pair 150. As used herein, cells having a “mirrored configuration” means that the cells 148 of a pair of cells are mirror images of each other. In such examples, each enlarged cell 148 in the pair 150 can be non-overlapping relative to the other cell 148 in the pair 150. That is, each enlarged cell 148 in the pair 150 can have an opposing shape relative to the other cell 148 in the pair 150 that does not coincide when one is superimposed on the other. In other examples, the enlarged cells 148 of each pair 150 can be overlapping or congruent, meaning that the cells 148 coincide with each other when one is superimposed on the other.

In some examples, each of the discontinuous cells 142 in the outflow row 140a can extend from a respective pair 150 of mirrored cells 148 in the second row 140b. As the commissure-supporting cells 122 are disposed circumferentially between respective pairs 150 of the enlarged cells 148 (discussed above), each of the commissure-supporting cells 122 can therefore be aligned with or disposed within one of the gaps 146 defined by the discontinuous cells 142 and the enlarged cells 148. In some examples, the commissure-supporting cells 122 are offset from the outflow end of the frame 102 defined by the apices 144 of the discontinuous cells 142 (discussed further below). In some examples, at least the outflow portion 154 of each of the commissure-supporting cells 122 can be disposed at an oblique angle relative to the longitudinal axis of the frame 102 and angled inward toward the longitudinal axis of the frame 102. In some examples, the outflow portion 154 of each of the commissure-supporting cells 122 can be disposed at a greater angle or can be angled inward toward the longitudinal axis to a greater degree than the discontinuous cells 142. In some examples, the outflow portion 154 of each of the commissure-supporting cells 122 can be angled inward at an oblique angle in a range of 5° to 35° relative to the longitudinal axis of the frame. In some examples, the outflow portion 154 of each of the commissure-supporting cells 122 can be angled inward at an oblique angle in a range of 5° to 25° relative to the longitudinal axis of the frame. For example, the commissure-supporting cells 122 can be angled inward at a 25° angle relative to the longitudinal axis of the frame. In some examples, the outflow portions 154 of the commissure-supporting cells 122 can be angled inward toward the longitudinal axis to an equal or lesser degree than the discontinuous cells 142. In some examples, the outflow portions of the of the commissure-supporting cells can be parallel to or generally parallel to the longitudinal axis of the frame or angled away from the longitudinal axis of the frame.

In some examples, the row of cells 140c is upstream relative to the row 140b and can include generally diamond-shaped or curved diamond-shaped cells 158, which can each be attached on either side to adjacent cells 158 within the row 140c. In some examples, the row of cells 140d is upstream relative to the row 140c and can include generally diamond-shaped or curved diamond-shaped cells 160, which can each be attached on either side to adjacent cells 160 within the row 140d. The apices 162 of the cells 160 can define the inflow end of frame 102. As discussed above, the inflow portion 136 of the frame 102 can have a flared configuration. In some examples, the flared configuration can be formed by inflow portions 164 of the cells 160 being angled outward from the longitudinal axis of the frame 102. For example, the inflow portions 164 of the cells 160 can be angled outward at an oblique angle in a range of 15° to 45° relative to the longitudinal axis of the frame 102.

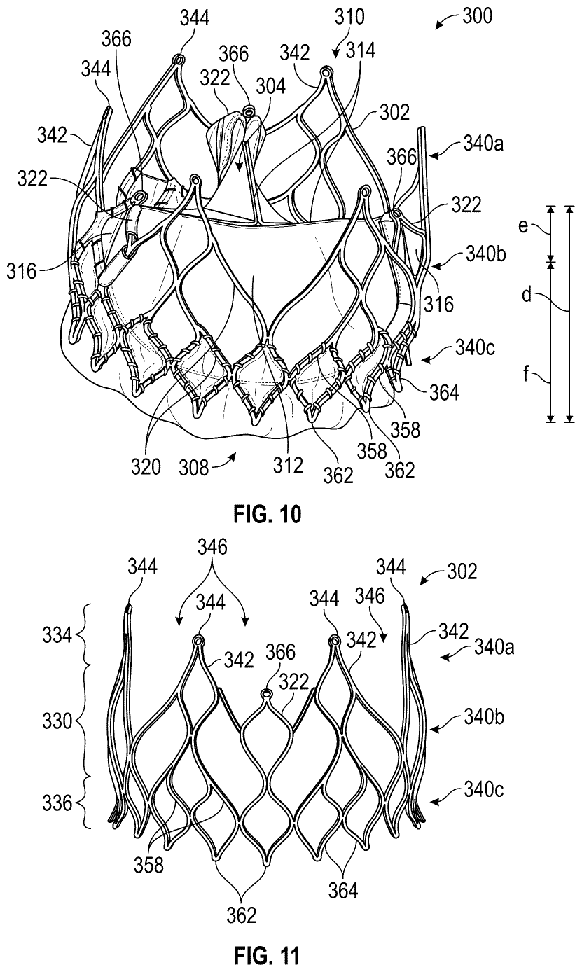

In the present example, the frame 102 includes twenty-four inflow apices 162 and six outflow apices 144, and thereby has a ratio of a number of inflow apices to a number of outflow apices of four to one (4:1). In some examples, the frame 102 can include more or fewer apices 162, 144 and have a four to one (4:1) ratio of a number of inflow apices to a number of outflow apices (such as, for example, twenty inflow apices and five outflow apices). In other examples, such as in the frame 302 shown in FIGS. 10-11 and discussed below, a frame can include a different ratio of inflow apices to outflow apices, such as, for example three to one (3:1).

Returning to FIG. 1, the frame 102 can have a total height a from the inflow end (defined by the apices 162 of the cells 160 in row 140d) to the outflow end (defined by the apices 144 of the discontinuous cells 142 in the row 140a). Also shown in FIG. 1, the outflow end apices 166 of the commissure-supporting cells 122 (in the row 140b) can be axially offset from the apices 144 of the discontinuous cells 142 by an offset distance b. The valvular structure 104 (including skirts 106, 128) is attached to and extends between the inflow end 108 of the frame 102 and the outflow portion of the commissure-supporting cells 122, which can have a distance c therebetween. In some examples, the distance c can generally correspond to a height of the valvular structure 104. As the commissure-supporting cells 122 are axially offset from the outflow end of the frame, the commissures 116 and the outflow edge portions 114 of the leaflets 112 can also be axially offset from the outflow end 110 of the frame 102. In some examples, the height a is in a range of 15 mm to 32 mm. In some examples, the offset distance b is in a range of 3 mm to 15 mm (such as, for example, a range of 3 mm to 7 mm). In some examples, the distance c is in a range of 12 mm to 25 mm. In some examples, a ratio of a height of the frame (a) to the distance c can be in a range of 1.1 to 2.7. In some examples, a ratio of a height of the frame (a) to the offset distance (b) can be in a range of 2.1 to 10.7. In one specific example, the height a is 25 mm, the offset distance b is 3 mm, and the distance c is 22 mm.

As illustrated in FIG. 7A, the prosthetic valve 100 can be implanted within a native valve (the aortic valve in the illustrated example) such that at least portions of the intermediate portion 130 and the inflow end portion 136 of the frame 102 are positioned within the aortic annulus 28, a portion of the intermediate portion 130 and the outflow end portion 134 extend above the aortic annulus into the Valsalva's sinuses 56, and the flared inflow apices 162 extend below the aortic annulus 28. The prosthetic valve 100 can be retained within the native valve by the radial outward force of the frame 102 against the surrounding tissue of the aortic annulus 28 as well as the geometry of the geometry or shape of the frame 102. For example, the bulging intermediate portion 130 and the flared inflow end portion 136, which at least partially extends radially outward beyond the aortic annulus 28, can resist against or limit or prevent axial dislodgement or displacement of the prosthetic valve 100 in the upstream and downstream directions (toward and away from the aorta). In some examples, the prosthetic valve 100 can be deployed within the native annulus 28 with the native leaflets 58 folded upwardly and compressed between the outer surface of the frame 102 and the walls of the Valsalva sinuses, as depicted in FIG. 7A. Depending on the condition of the native leaflets 58, in some examples, it may be desirable to excise the native leaflets 58 prior to implanting the prosthetic valve 100. As noted above, only a portion of the frame 102 is shown in FIG. 7A for illustrative purposes, however, the implanted prosthetic valve 100 can have the configuration shown in FIGS. 1 and 2.

FIGS. 7B-7C shown an exemplary delivery apparatus that can be used for delivery and/deployment of a self-expandable prosthetic valve. For example, FIGS. 7B-7C illustrates a delivery apparatus 10, according to one example, which is adapted to advance a prosthetic heart valve 30 (or other prosthetic heart valve), through a patient's vasculature and/or to deliver the prosthetic heart valve 30 to an implantation site (for example, native heart valve) within a patient's body. The prosthetic heart valve 30 can be mounted on, retained within, and/or releasably coupled to a distal end portion of the delivery apparatus 10.

The prosthetic heart valve 30 is representative of the prosthetic heart valve 100 (or other prosthetic valves disclosed herein, such as the prosthetic valves 200, 300, 400, 500 discussed below) and therefore can be identical to any of the prosthetic valves disclosed herein.

The delivery apparatus 10 in the illustrated example generally includes the handle 16, a first elongated shaft 17 (which comprises an outer shaft in the illustrated example) extending distally from the handle 16, a second shaft 18 extending from the handle and coaxially though the first shaft 17, and a third shaft 20 (which comprises an inner shaft in the illustrated example) extending from the handle and coaxially through the second shaft 18. A nose cone 22 can connected to the distal end portion of the inner shaft 20.

As discussed above, in some examples, the prosthetic valve 30 can be self-expanding. That is, the prosthetic valve 30 can be configured to self-expand from a radially compressed, delivery state to a radially expanded state. When configured to be self-expanding, the frame of the prosthetic valve 30 can be constructed from a shape-memory material (for example, Nitinol) that biases the prosthetic valve 30 towards a radially expanded state. In such examples, the struts can be configured and connected to each other such that the frame has inherent resiliency that causes the frame to self-expand to a radially expanded state when released from a constraining member (for example, a delivery sheath or capsule, discussed further below). Further, the prosthetic valve 30 can be shape set in the radially expanded state so that the prosthetic valve 30 returns to the shape set radially expanded state when released from a restraining mechanism (for example, lasso, sheath, capsule, and/or outer skirt, etc.).

In some examples, the shaft 17 can have a distal end portion 24 sized to house the prosthetic valve in its radially compressed, delivery state during delivery of the prosthetic valve through the patient's vasculature. In this manner, the distal end portion 24 functions as a delivery sheath or capsule for the prosthetic valve during delivery, and as such may be referred to herein as a capsule 24. In some examples, deployment or release of the prosthetic valve 30 from the capsule 24 results in transition of the valve from the radially compressed state to a fully radially expanded state. Deployment of the prosthetic valve 30 from the capsule 24, either by retracting the capsule proximally relative to the prosthetic valve or advancing the prosthetic valve distally relative to the capsule, allows the prosthetic valve to self-expand from a radially compressed state (FIG. 7B) to a radially expanded state. FIG. 7C shows the prosthetic 30 partially expanded as it is being deployed from the capsule 24.

The prosthetic valve 30 can be releasably connected to the distal end portion of the second shaft 18, such as with tethers (not shown) that are threaded through apertures in the apices 144 and/or apices 166 of the frame of the valve and coupled to the distal end portion of the second shaft 18 or to a suture retention member (not shown) coupled to the second shaft 18. Once the prosthetic valve is deployed from the capsule 24 and radially expands, the tethers can be removed from the frame of the prosthetic valve to disconnect the prosthetic valve from the delivery device. Further details regarding the structure and techniques for releasably connecting the prosthetic valve 30 to the delivery device with tethers is disclosed in U.S. Pat. No. 9,867,700, which is incorporated herein by reference. Other techniques and mechanisms known in the art can be used to form a releasably connection between the prosthetic valve 30 and the delivery device 10 can be used.

The handle 16 of the delivery apparatus 10 can include one or more control mechanisms (for example, knobs or other actuating mechanisms) for controlling different components of the delivery apparatus 10 in order to implant the prosthetic heart valve 30. For example, in the illustrated example the handle 16 can comprise first and second knobs 11 and 12, respectively. In some examples, the delivery apparatus 10 can include more or fewer knobs. In other examples, the delivery apparatus 10 can include other types of actuators (for example, buttons or switches).

In some examples, the knob 11 can be configured to produce axial movement of the shaft 17 relative to the prosthetic heart valve 30 in the distal and/or proximal directions in order to deploy the prosthetic valve 30 from the capsule 24 once the prosthetic valve 30 has been advanced to a location at or adjacent the desired implantation location within the patient's body. For example, actuating the first knob 11 in a first direction (for example, clockwise) can retract the shaft 17 proximally relative to the prosthetic heart valve 30 (see FIG. 7C) and actuation of the first knob 11 in a second direction (for example, counter-clockwise) can advance the shaft 17 distally. The first knob 11 can be actuated by rotating the knob as indicated above, or by sliding or moving the knob axially, such as by pulling and/or pushing the knob.

In some examples, the knob 12 can be configured to adjust the curvature of the delivery end portion of the delivery device 10 to facilitate steering of the delivery device through the vasculature of the patient. For example, the knob 12 can be operatively coupled to a steering mechanism housed in the handle 16 and a pull wire that extends from the steering mechanism through the outer shaft 18. The pull wire can have a distal end fixed at a location near the distal end of the outer shaft such that adjusting the tension of the pull wire is effective to adjust the curvature of the shafts 17, 18, and 20. Further details regarding steering mechanisms for delivery devices are disclosed in U.S. Publication No. 2013/0030519, which is incorporated herein by reference.

It will be appreciated that the delivery apparatus disclosed herein can include other features and/or the prosthetic valves disclosed herein can be utilized with other types of delivery apparatus, such as those described in U.S. Provisional Patent Application No. 63/179,766 and U.S. Pat. Nos. 8,652,202; 9,155,619; and 9,867,700, which are incorporated by reference herein, or others, for example, balloon-expandable delivery apparatus, such as those described in U.S. Provisional Patent Application No. 63/374,343, previously incorporated by reference herein.

The prosthetic valve 100 can address one or more of the issues of various known prosthetic valves discussed above. For example, the enlarged cells 148 and the gaps 146 in the frame 102 can enable coronary access after implantation of the prosthetic valve 100 (such as, for example, enabling insertion or manipulation of tools, wires, or catheters therethrough in post-implantation procedures). In another example, the prosthetic valve 100 can have an overall shorter height relative to some known prosthetic valves, which can also improve coronary access after implantation of the prosthetic valve. The shorter height of the valve 100 can additionally limit or prevent contact with the AV node when the prosthetic valve is implanted in a native tricuspid valve.

In another example, the enlarged cells 148, the gaps 146, and the discontinuous cells 142 of the frame 102 can enable the prosthetic valve 100 to have an overall reduced bulk or profile when the valve 100 is a radially compressed state relative to other prosthetic valves of a same or similar overall height. The reduced profile in the radially compressed state can improve or ease transcatheter delivery of the prosthetic valve 100.

In another example, the discontinuous cells 142 in the outflow row 140a can prevent tissue overhang when the prosthetic valve 100 is implanted within a native valve. For example, as can be seen in FIG. 7A, the discontinuous cells 142 in the outflow portion 134 of the frame 102 can capture and compress the edges of the native leaflets 58 against the walls of the Valsalva sinuses and prevent the leaflet edges from extending over the outflow end 110 of the prosthetic valve 100. In another example, the outflow end of the valvular structure 104 (for example, the commissures 116 and the outflow edge portions 114 of the leaflets 112) is offset from the outflow end of the frame 102 (defined by the outflow apices 144) via attachment of the leaflets 112 to the commissure-supporting cells 122. The offset of the outflow end of the valvular structure 104 can, for example, provide an open or unobstructed area directly downstream of the outflow end of the valvular structure 104. In another example, an overall height of valvular structure can be shorter relative to valvular structures of some known prosthetic valves. One or more of the foregoing features can improve hemodynamics of prosthetic valve 100. For example, one or more of the foregoing features can improve turbulence to assist in washing of the leaflets, minimize stagnant blood flow, and/or prevent or limit thrombosis.

In yet another example, the prosthetic valve 100 can have improved recapturability or retrievability relative to known prosthetic valves. For example, if necessary to remove or reposition the prosthetic valve during an implantation procedure, the reduced number of outflow apices (relative to a number of inflow apices) can be more easily recaptured into a capsule at a distal end of a delivery apparatus (for example, the capsule 24 of the delivery apparatus 10 discussed above) relative to a prosthetic valve having a greater or equal number of outflow apices (relative to a number of inflow apices). In another example, the inward angle of the discontinuous cells relative to the longitudinal axis of the frame can assist in recapture of the prosthetic valve into a capsule of a delivery apparatus. In another example, the inward angle of the commissure-supporting cells relative to the longitudinal axis of the frame can assist in causing the valvular structure and the body of the frame in the intermediate portion to “dive” inward or collapse inward during recapture. In yet another example, the enlarged cells, which have a reduced number of struts or a greater open area of the cell relative to non-enlarged cells, can reduce a compressive force required for radial compression and recapture of the prosthetic valve.

In some examples, other prosthetic valves disclosed herein can include a frame having a modified or different configuration relative to the frame 102 and can have one or more of the foregoing benefits or advantages over various known prosthetic valves. For example, FIGS. 8-9 illustrate an exemplary annular frame 202 that can be included in a self-expanding or balloon-expandable prosthetic valve and can have a valvular structure similar to the valvular structure 104 disposed therein and attached thereto. The frame 202 can be made of a shape memory material (for example, Nitinol) or one or more of the plastically-expandable materials discussed above with respect to the frame 102. The frame 202 can have cells (for example, rows of cells), which can be formed by interconnected ones of struts 220 of the frame 202.

The frame 202 can include an outflow end portion 234, an inflow end portion 236, and an intermediate portion 230 disposed therebetween. In some examples, the intermediate portion 230 has an overall bulging or outward curved shape that tapers towards each of the outflow end portion 234 and the inflow end portion 236. In some examples, the inflow end portion 236 can flare outward relative to a longitudinal axis of the prosthetic valve. In some examples, the inflow end portion 236 includes a row of cells 240d and the intermediate portion 230 includes rows of cells 240c and 240b, which can have a similar configuration to the row of cells 140d of inflow end portion 136 and the rows of cells 140c and 140b of the intermediate portion 130 in the frame 102, respectively.

Different from the frame 102, the outflow end portion 234 can be angled outward from the longitudinal axis of the prosthetic valve 100. In some examples, the outflow end portion 234 can include a (first) outflow row of cells 240a including a plurality of discontinuous cells 242. In some examples, the outflow end of the frame 202 is defined by outflow apices 244 of the discontinuous cells 242 in the outflow row 240a. In some examples, each cell 242 in the outflow row 240a can have a generally diamond-shaped or a curved diamond-shaped configuration that is unattached to other cells 242 of the outflow row. As the cells 242 are unattached to each other, the outflow row of cells 240a can (at least in part) define a plurality of gaps 246 in the frame 202 between adjacent ones of the discontinuous cells 242. In some examples, the gaps 246 are further defined in part by one or more cells in the row 240b. As shown in FIGS. 8-9, in some examples, the discontinuous cells 242 can be angled outward from the longitudinal axis of the frame 202. In some examples, the discontinuous cells 242 can be disposed outward at an oblique angle in a range of 20° to 30° relative to the longitudinal axis of the frame. In some examples, the outflow apices 244 can be bent or angled inward, such as, for example, being disposed inward at an oblique angle in a range of 10° to 15° relative to the longitudinal axis of the frame In some examples, each of the outflow apices 244 can include an aperture or other structural feature (for example, a tab) configured for attachment or coupling of the prosthetic valve to a delivery apparatus. Thus, in such examples, the outflow end portion 234 can be a flared portion of the frame 202.

As discussed above, other portions of the frame 202 can have a similar configuration to the frame 102. For example, the row 240b can include commissure-supporting cells 222 are disposed circumferentially between respective pairs 250 of the enlarged cells 248, each of the commissure-supporting cells 222 can therefore be aligned with or disposed within one of the gaps 246 defined by the discontinuous cells 242 and the enlarged cells 248. In some examples, the commissure-supporting cells 222 are offset from the outflow end of the frame 202 defined by apices 244 of the discontinuous cells 242. In some examples, at least the outflow portion 254 of each of the commissure-supporting cells 222 can be disposed at an oblique angle relative to the longitudinal axis of the frame 102 and angled inward toward the longitudinal axis of the frame 102. In some examples, the outflow portion 254 of each of the commissure-supporting cells 222 can be angled inward toward the longitudinal axis of the frame. In some examples, the outflow portion 254 of each of the commissure-supporting cells 222 can be angled inward at an oblique angle in a range of 5° to 35° relative to the longitudinal axis of the frame. In some examples, the outflow portion 254 of each of the commissure-supporting cells 222 can be angled inward at an oblique angle in a range of 5° to 25° relative to the longitudinal axis of the frame. In some examples, the outflow portions of the of the commissure-supporting cells can be parallel to or generally parallel to the longitudinal axis of the frame or angled away from the longitudinal axis of the frame.

A prosthetic valve including the frame 202 can have one or more of the advantages of the prosthetic valve 100 (discussed above). Further, in some examples, the discontinuous cells 242 in the outflow row 240a that are angled outward from longitudinal axis of the prosthetic valve (thereby creating a flared outflow portion 234) can provide an outward radial force or spring force on surrounding tissue when the prosthetic valve is implanted within a native valve. For example, the discontinuous cells 242 can prevent tissue overhang when the prosthetic valve including the frame 202 is implanted within a native valve by compressing the edges of the native leaflets against the walls of the Valsalva sinuses and prevent the leaflet edges from extending over the outflow end of the prosthetic valve. In another example, the flared configuration of the inflow end can resist against or limit or prevent axial dislodgement or displacement of the prosthetic valve in the upstream and downstream directions (toward and away from the aorta).

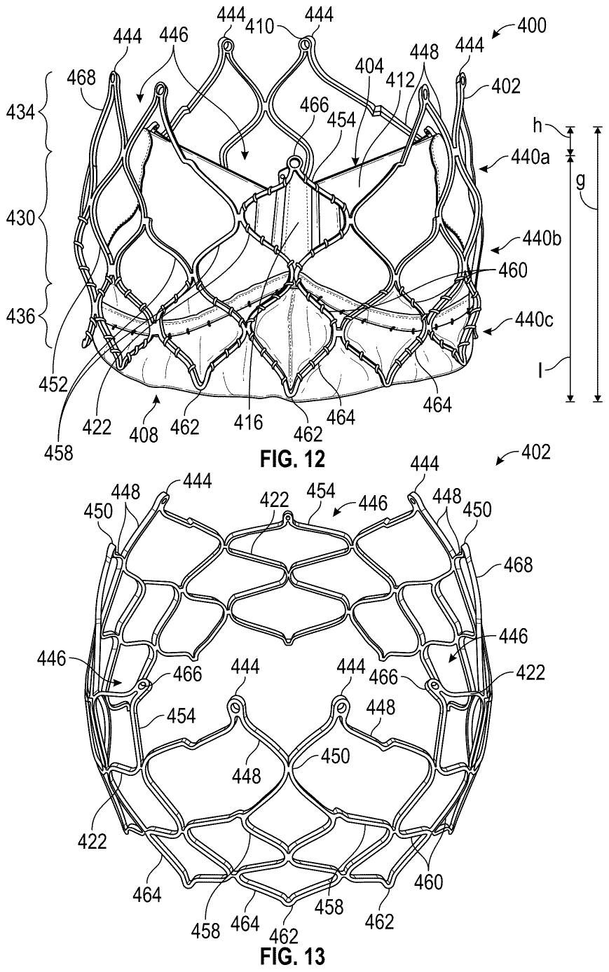

FIGS. 11-12 illustrate another exemplary prosthetic valve 300 including an annular frame 302 that can be included in a self-expanding or balloon-expandable prosthetic valve and can have a valvular structure 304 similar to the valvular structure 104 disposed therein and attached thereto. The frame 302 can be made of a shape-memory material or one or more of the plastically-expandable materials discussed above, and rows of cells can be formed by interconnected ones of struts 320 of the frame 302. Although only one side of the frame 302 is depicted in FIG. 11, it should be appreciated that the frame 302 forms an annular structure having an opposite side that is substantially identical to the portion shown in FIG. 11.

The frame 302 can include an outflow end portion 334 and an inflow end portion 336 on opposing ends of an intermediate portion 330. In some examples, the intermediate portion 330 has an overall bulging or outward curved shape that tapers towards each of the outflow end portion 334 and the inflow end portion 336. In some examples, the inflow end portion 336 can flare outward relative to a longitudinal axis of the prosthetic valve. In some examples, the outflow end portion 334 includes a row of cells 340a and the intermediate portion 330 includes a rows of cells 340b, which can have a similar configuration to the row of cells 140a in the outflow end portion 134 and the row of cells 140b the intermediate portion 130 of the frame 102.

Different from the frame 102, a row of cells 340c that is upstream of the row of cells 340b can form the inflow portion 336 of the frame 302. Thus, in some examples, the frame 302 can have one less row of cells than the frames 102, 202. In some examples, the row of cells 340c can include generally diamond-shaped or curved diamond-shaped cells 358, which can each be attached on either side to adjacent cells 358 within the row 340c. Apices 362 of the cells 358 can define the inflow end of frame 102. The inflow portion 336 of the frame 302 can have a flared configuration. In some examples, the flared configuration can be formed by inflow portions 364 of the cells 358 being angled outward from the longitudinal axis of the frame 302. For example, the inflow portions 364 of the cells 358 can be angled outward at an oblique angle in a range of 15° to 45° relative to the longitudinal axis of the frame 302.

In the present example, the frame 302 includes eighteen inflow apices 362 and six outflow apices 344 formed by discontinuous cells 342 in the row 340a, and thereby has a ratio of a number of inflow apices to a number of outflow apices of three to one (3:1). In some examples, the frame 302 can include more or fewer apices 362, 344 and have a three to one (3:1) ratio of a number of inflow apices to a number of outflow apices (such as, for example, twenty-one inflow apices and seven outflow apices).

As can be seen in FIG. 10, the frame 302 can have a total height d from the inflow end (defined by the apices 362 of the cells 358 in row 340c) to the outflow end (defined by the apices 344 of the discontinuous cells 342 in the row 340a). Similar to the frame 102, in some examples, circumferentially arranged commissure-supporting cells 322 (in the row 340b) can be aligned with gaps 346 in the frame 302. In some examples, outflow end apices 366 of the commissure-supporting cells 322 can be axially offset from the apices 344 (defining the outflow end 310 of the prosthetic valve 300) by an offset distance e. The valvular structure 104 is attached to and extends between the inflow end 308 of the frame 302 and the outflow portions of the commissure-supporting cells 322, which can have a distance f therebetween. In some examples, the distance f can generally correspond to a height of the valvular structure 104. As the commissure-supporting cells 322 are axially offset from the outflow end of the frame, commissures 316 and outflow edge portions 314 of leaflets 312 in the valvular structure 304 can also be axially offset from the outflow end 310 of the frame 302 and the prosthetic valve 300. In some examples, the height d is in a range of 15 mm to 32 mm. In some examples, the offset distance e is in a range of 3 mm to 15 mm (such as, for example, a range of 5 mm to 15 mm). In some examples, the distance f is in a range of 10 mm to 17 mm. In some examples, a ratio of a height of the frame (d) to the distance f can be in a range of 1.1 to 3.2. In some examples, a ratio of a height of the frame (d) to the offset distance (e) can be in a range of 2 to 6.4. In one specific example, the height d is 25 mm, the offset distance e is 12 mm, and the distance f is 13 mm.

As discussed above, the prosthetic valve 300 including the frame 302 can have one or more of the foregoing advantages discussed above with respect to the prosthetic valve 100 including the frame 102. Additionally, the prosthetic valve 300 can have an overall shorter height relative to some known prosthetic valves, which can improve coronary access after implantation of the valve and/or can limit or prevent contact with the AV node when the prosthetic valve is implanted in a native tricuspid valve.

Turning to FIGS. 12-13 and 14-15, exemplary prosthetic valves 400 and 500, respectively, are shown and described. Different from other prosthetic valves disclosed herein (for example, the prosthetic valves 100, 300), the outflow ends of the prosthetic valves 400 and 500 can be defined by outflow portions of pairs of cells (for example, outflow portions of pairs of mirrored cells).

As shown in FIGS. 12-13, the prosthetic valve 400 can include an annular frame 402. The prosthetic valves 400 can be a self-expanding or balloon-expandable prosthetic valve and can have a valvular structure 404 similar to the valvular structure 104 disposed therein and attached thereto. The frame 402 can be made of a shape-memory material (for example, Nitinol) or one or more of the plastically-expandable materials discussed above, and rows of cells can be formed by interconnected ones of struts 420 of the frame 402.

Specifically, the frame 402 can include an outflow end portion 434 and an inflow end portion 436 on opposing ends of an intermediate portion 430. In some examples, the intermediate portion 430 has an overall bulging or outward curved shape that tapers towards each of the outflow end portion 434 and the inflow end portion 436. As in illustrated example, the bulge or outward curvature of the intermediate portion 430 can be to a lesser degree than that other frames disclosed herein (for example, the frames 102, 202, 302). In some examples, the bulge or outward curvature of the intermediate portion 430 can be to a same degree or a greater degree than that other frames disclosed herein. In some examples, the inflow end portion 436 can flare outward relative to a longitudinal axis of the prosthetic valve.

In some examples, a (first) row 440a of cells includes enlarged cells 448 arranged in mirrored pairs 450, commissure-supporting cells 422, and cells 452 disposed between the enlarged cells 448 in each of the pairs 150. The outflow end portion 434 of the frame 402 can include outflow portions of the mirrored pairs 450 of enlarged cells 448 in the first row 440a. The intermediate portion 430 can include inflow portions of the mirrored pairs 450 of enlarged cells 448, the commissure-supporting cells 422, and the cells 452 in the row 440a, as well as a (third) row of cells 440b upstream of the row 440a. The inflow end portion 136 can include a (fourth) row of cells 140d.

As discussed above, in some examples, an outflow end 410 of the frame 402 and the prosthetic valve 400 can be defined by outflow apices 444 of the mirrored pairs 450 of enlarged cells 448 in the first row 440a. As the outflow portions 468 (including the outflow apices 444) of the enlarged cells 448 extend downstream of the commissure-supporting cells 422, the outflow portions 468 of the enlarged cells 448 can define a plurality of gaps 446 in the frame 402. In some examples, the outflow portions 468 of the enlarged cells 448 can be angled inward toward the longitudinal axis of the frame 402. For example, the outflow portions 468 of the enlarged cells 448 can be angled inward at an oblique angle in a range of 1° to 15° relative to the longitudinal axis of the frame. In other examples, the outflow portions 468 of the enlarged cells 448 can be parallel relative to the longitudinal axis of the frame 402, or can be angled outward. In some examples, each of the outflow apices 444 can include an aperture or other structural feature (for example, a tab) configured for attachment or coupling of the prosthetic valve 400 to a delivery apparatus.

As can be seen in FIGS. 12-13, the commissure-supporting cells 422 can have a generally diamond-shaped or a curved diamond-shaped configuration and can be disposed circumferentially between respective pairs 450 of the enlarged cells 448. In some examples, the commissure-supporting cells 422 can be generally aligned (along the longitudinal axis of the frame) with the cells 452 in the row 440a. In other examples, the commissure-supporting cells can be offset (for example, offset in either a downstream direction or an upstream direction) relative to the cells 452.

In some examples, the enlarged cells 448 can be larger than the cells 422, 452. In some examples, the enlarged cells 448 can be larger than all other cells in the frame 402. In some examples, each of the enlarged cells 448 can have a shape generally corresponding to two substantially diamond-shaped or curved diamond-shaped cells having a central strut removed therebetween. In such examples, each of the enlarged cells 448 can be approximately two times the size of other cells in the frame 402 (for example, each of the enlarged cells 448 can be approximately two times the size of one of the cells 452). In some examples, of the enlarged cells 448 can be in a range of 1.1 to 5, such as a range of 1.5 to 4, 1.8 to 3, 2 to 2.5, etc., times larger than other cells in the frame 402.

In some examples, each of the enlarged cells 448 can have an elongated, curved diamond shape (for example, S-shape), where the curved diamond is elongated between the apex 444 and outflow portions of cells in the of the row 440b. In some examples, the enlarged cells 448 can be joined at a center point of the pair 450, and each enlarged cell 448 can have a mirrored configuration around the center point relative to the other enlarged cell 448 in the pair 450. In such examples, each enlarged cell 448 in the pair 450 can be non-overlapping relative to the other cell 448 in the pair 450. That is, each enlarged cell 448 in the pair 450 can have an opposing shape relative to the other cell 448 in the pair 450 that does not coincide when one is superimposed on the other. In other examples, the enlarged cells 448 in the pair 450 can be overlapping (congruent).

As the commissure-supporting cells 422 are disposed circumferentially between respective pairs 450 of the enlarged cells 448, each of the commissure-supporting cells 422 can therefore be aligned with or disposed within one of the gaps 446 defined by the outflow portions of the enlarged cells 448. In some examples, the commissure-supporting cells 422 are offset from the outflow end of the frame 402 defined by the apices 444 of the enlarged cells 448 (discussed further below). In some examples, at least the outflow portion 454 of each of the commissure-supporting cells 422 can be disposed at an oblique angle relative to the longitudinal axis of the frame 402 and angled inward toward the longitudinal axis of the frame 402. In some examples, the outflow portion 454 of each of the commissure-supporting cells 422 can be disposed at a greater angle or can be angled inward toward the longitudinal axis to a greater degree than the outflow portions of the enlarged cells 448. In some examples, the outflow portion 454 of each of the commissure-supporting cells 422 can be angled inward at an oblique angle in a range of 20° to 30° relative to the longitudinal axis of the frame. In some examples, the outflow portions 454 of the commissure-supporting cells 422 can be angled inward toward the longitudinal axis to an equal or lesser degree than the outflow portions of the enlarged cells 448. In some examples, the outflow portions of the of the commissure-supporting cells can be parallel to or generally parallel to the longitudinal axis of the frame or angled away from the longitudinal axis of the frame.

As discussed above, the row of cells 440b can be upstream relative to the row 440a and can include generally diamond-shaped or curved diamond-shaped cells 458, which can each be attached on either side to adjacent cells 458 within the row 440b. In some examples, the row of cells 440c is upstream relative to the row 440b and can include generally diamond-shaped or curved diamond-shaped cells 460, which can each be attached on either side to adjacent cells 460 within the row 440c. The apices 462 of the cells 460 can define the inflow end 408 of frame 402 and the prosthetic valve 400. As discussed above, the inflow portion 436 of the frame 402 can have a flared configuration. In some examples, the flared configuration can be formed by inflow portions 464 of the cells 460 being angled outward from the longitudinal axis of the frame 402. For example, the inflow portions 464 of the cells 460 can be angled outward at an oblique angle in a range of 15° to 45° relative to the longitudinal axis of the frame 402.

In the present example, the frame 402 includes twelve inflow apices 462 and six outflow apices 444, and thereby has a ratio of a number of inflow apices to a number of outflow apices of two to one (2:1). In some examples, the frame 402 can include more or fewer apices 462, 444 and have a two to one (2:1) ratio of a number of inflow apices to a number of outflow apices (such as, for example, sixteen inflow apices and eight outflow apices). In other examples, a frame can include a different ratio of inflow apices to outflow apices, such as, for example three to one (3:1) or four to one (4:1).

As shown in FIG. 12, the frame 402 can have a total height g from the inflow end (defined by the apices 462 of the cells 460 in row 440c) to the outflow end (defined by the apices 444 of the enlarged cells 448 in the row 440a). Also shown in FIG. 12, the outflow end apices 466 of the commissure-supporting cells 422 (in the row 440a) can be axially offset from the apices 444 of the enlarged cells 448 by an offset distance h. The valvular structure 404 is attached to and extends between the inflow end 408 of the frame 402 and the outflow portions of the commissure-supporting cells 422, which can have a distance i therebetween. In some examples, the distance i can generally correspond to a height of the valvular structure 404. As the commissure-supporting cells 422 are axially offset from the outflow end of the frame, the commissures 416 and the outflow edge portions 414 of the leaflets 412 of the valvular structure 404 can also be axially offset from the outflow end 410 of the frame 402 and the prosthetic valve 400. In some examples, the height g is in a range of 15 mm to 32 mm. In some examples, the offset distance h is in a range of 3 mm to 15 mm (such as, for example, a range of 5 mm to 15 mm). In some examples, the distance i is in a range of 10 mm to 17 mm. In some examples, a ratio of a height of the frame (g) to the distance i can be in a range of 1.1 to 3.2. In some examples, a ratio of a height of the frame (g) to the offset distance (h) can be in a range of 2 to 6.4. In one specific example, the height g is 25 mm, the offset distance e is 12 mm, and the distance f is 13 mm.

The prosthetic valve 400 can be implanted within a native valve (such as, for example, within an aortic valve. In some examples, the prosthetic valve 400 can be implanted in a similar manner as the implanted prosthetic valve 100 illustrated in FIG. 7A. In some examples, the prosthetic valve 400 can be implanted can be implanted in other native valves, such as, for example, a tricuspid valve. In some examples, the prosthetic valve 400 can be implanted in a native valve utilizing the exemplary delivery apparatus 10 illustrated in FIG. 7B. In some examples, prosthetic valve 400 can be implanted utilizing other delivery apparatus, such as those described in U.S. Pat. No. 9,867,700, which is incorporated by reference herein, and U.S. Provisional Patent Application No. 63/374,343 , previously incorporated herein.

The prosthetic valve 400 can address one or more of the issues of various known prosthetic valves discussed above. For example, the enlarged cells 448 and the gaps 446 in the frame 402 can enable coronary access after implantation of the prosthetic valve 400 (such as, for example, enabling insertion or manipulation of tools, wires, or catheters therethrough in post-implantation procedures). In another example, the prosthetic valve 400 can have an overall shorter height relative to some known prosthetic valves, which can also improve coronary access after implantation of the prosthetic valve. The shorter height of the valve 400 can additionally limit or prevent contact with the AV node when the prosthetic valve is implanted in a native tricuspid valve.

In another example, the enlarged cells 448 and the gaps 446 of the frame 402 can enable the prosthetic valve 400 to have an overall reduced bulk or profile when the valve 400 is a radially compressed state relative to other prosthetic valves of a same or similar overall height. The reduced profile in the radially compressed state can improve or ease transcatheter delivery of the prosthetic valve 400.

In another example, the mirrored pairs 450 of enlarged cells 448 in the outflow row 440a can prevent tissue overhang when the prosthetic valve 400 is implanted within a native valve. For example, the mirrored pairs 450 of enlarged cells 448 in the outflow portion 434 of the frame 402 can capture and compress the edges of the native leaflets against the walls of the Valsalva sinuses and prevent the leaflet edges from extending over the outflow end 410 of the prosthetic valve 400. In another example, the outflow end of the valvular structure 404 (for example, the commissures 416 and the outflow edge portions of leaflets 412) is offset from the outflow end 410 of the frame 402 (defined by the outflow apices 444) via attachment of the leaflets 412 to the commissure-supporting cells 422. The offset of the outflow end of the valvular structure 404 can, for example, provide an open or unobstructed area directly downstream of the outflow end of the valvular structure 404. In another example, an overall height of valvular structure can be shorter relative to valvular structures of some known prosthetic valves. One or more of the foregoing features can improve hemodynamics of prosthetic valve 400. For example, one or more of the foregoing features can improve turbulence to assist in washing of the leaflets, minimize stagnant blood flow, and/or prevent or limit thrombosis.

In yet another example, the prosthetic valve 400 can have improved recapturability or retrievability relative to known prosthetic valves. For example, if necessary to remove or reposition the prosthetic valve during an implantation procedure, the reduced number of outflow apices (relative to a number of inflow apices) can be more easily recaptured into a capsule at a distal end of a delivery apparatus (for example, the capsule 24 of the delivery apparatus 10 discussed above) relative to a prosthetic valve having a greater or equal number of outflow apices (relative to a number of inflow apices). In another example, the inward angle of the outflow portions of the enlarged cells 448 relative to the longitudinal axis of the frame can assist in recapture of the prosthetic valve into a capsule of a delivery apparatus. In another example, the inward angle of the commissure-supporting cells 422 relative to the longitudinal axis of the frame can assist in causing the valvular structure and the body of the frame in the intermediate portion to “dive” inward or collapse inward during recapture. In yet another example, the enlarged cells, which have a reduced number of struts or a greater open area of the cell relative to non-enlarged cells, can reduce a compressive force required for radial compression and recapture of the prosthetic valve.

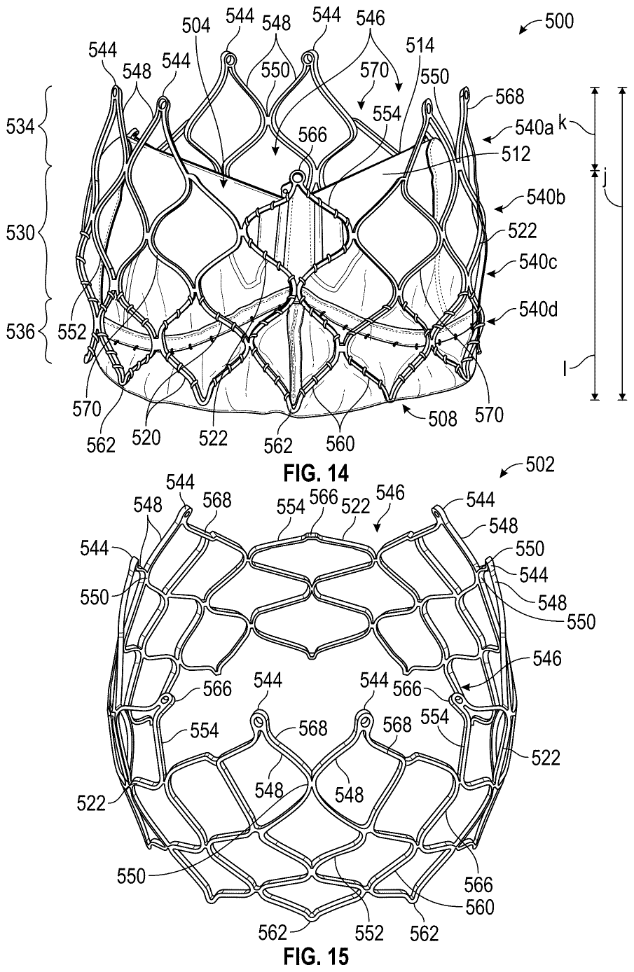

In some examples, other prosthetic valves disclosed can include a frame having a modified or different configuration relative to the frame 402 here that can have one or more of the foregoing benefits or advantages over various known prosthetic valves. For example, FIGS. 14-15 illustrate a prosthetic valve 500 including an exemplary annular frame 502 that can be included in a self-expanding or balloon-expandable prosthetic valve and can have a valvular structure 504 (which can be similar to the valvular structure 104) disposed therein and attached thereto. The frame 502 can be made of a shape-memory material (for example, Nitinol) or one or more of the plastically-expandable materials discussed above with respect to the frame 102, and cells (for example, rows of cells) can be formed by interconnected ones of struts 520 of the frame 502.

The frame 502 can include an outflow end portion 534, an inflow end portion 536, and an intermediate portion 530 disposed therebetween. In some examples, the intermediate portion 530 has an overall bulging or outward curved shape that tapers towards each of the outflow end portion 534 and the inflow end portion 536. In some examples, the inflow end portion 536 can flare outward relative to a longitudinal axis of the prosthetic valve. In some examples, the inflow end portion 536 includes a row of cells 540d and the intermediate portion 530 includes rows of cells 540c, which can have a similar configuration to the row of cells 440c of inflow end portion 436 and the row of cells 440b of the intermediate portion 430 in the frame 402.

Different from the frame 402, the outflow end portion 534 can comprise pairs 550 of cells 548, where the cells 548 can be generally diamond-shaped or curved diamond-shaped and can be similar in size or non-enlarged relative to other cells in the frame 502. In some examples, the cells 548 in each pair 550 can have an offset, curved diamond shape (for example, S-shape). In some examples, the cells 548 can be joined at a center point of the pair 550, and each cell 548 can have a mirrored configuration around the center point relative to the other cell 548 in the pair 550. In such examples, each cell 548 in the pair 550 can be non-overlapping relative to the other cell 548 in the pair 550. That is, each cell 548 in the pair 550 can have an opposing shape relative to the other cell 548 in the pair 550 that does not coincide when one is superimposed on the other. In other examples, each cell in the pair of cells at the outflow end can instead have a same shape as the other cell in the pair, wherein the shape of the cells coincide when one is superimposed on the other (that is, the cells of each pair are overlapping or congruent).

In some examples, an outflow end 510 of the frame 502 and the prosthetic valve 500 can be defined by outflow apices 544 of the pairs 550 of cells 548 in the first row 540a. As the outflow portions 568 (including the outflow apices 544) of the cells 548 extend downstream of commissure-supporting cells 522, the outflow portions 568 of the cells 548 can define a plurality of gaps 546 in the frame 502. In some examples, at least the outflow portions 568 of the cells 548 (for example, the outflow portion or an entirety of the cell) can be angled inward toward the longitudinal axis of the frame 502. For example, at least the outflow portions 568 of the cells 548 can be angled inward at an oblique angle in a range of 1° to 15° relative to the longitudinal axis of the frame 502. In other examples, the outflow portions 568 of the cells 548 can be parallel to the longitudinal axis of the frame 502. In some examples, each of the outflow apices 544 can include an aperture or other structural feature (for example, a tab) configured for attachment or coupling of the prosthetic valve 500 to a delivery apparatus.

Also different from the frame 402, the intermediate portion 530 can include (in addition to the row of cells 540c) a row of cells 540b. The row of cells 540b can include commissure-supporting cells 522, cells 552 disposed between cells 548 in each of the pairs 550, and cells 570 disposed between the cells 552 and the commissure-supporting cells 522. Each of the cells in the row 540b can have a generally diamond-shaped or a curved diamond-shaped configuration. Each of the commissure-supporting cells 522 can be disposed circumferentially between respective pairs 550 of the cells 548.

In some examples, the outflow end 510 of the frame 502 is defined by outflow apices 544 of the cells 548 in the outflow row 540a. As the outflow portions (including the outflow apices 544) of the cells 548 extend downstream of the commissure-supporting cells 522, at least the outflow portions 568 of the cells 548 can define a plurality of gaps 546 in the frame 502.

As discussed above, other portions of the frame 502 can have a similar configuration to the frame 402. For example, cells in the inflow row of cells 540d can be similar to cells in the inflow row 440c of the frame 402 and cells downstream of the inflow row 540d can be similar to cells in the row 440b in the frame 402. In another example, as the commissure-supporting cells 522 are disposed circumferentially between respective pairs 550 of the cells 548, each of the commissure-supporting cells 522 can be aligned with or disposed within one of the gaps 546 (similar to the commissure-supporting cells 422 and the gaps 446).

In some examples, the commissure-supporting cells 522 are offset from the outflow end 510 of the frame 502 defined by apices 544 of the cells 548. As shown in FIG. 14, the frame 502 can have a total height j from the inflow end 508 (defined by apices 562 of cells 560 in row 540d) to the outflow end 510 (defined by the apices 544 of the cells 548 in the row 540a). Also shown in FIG. 14, the outflow end apices 566 of the commissure-supporting cells 522 (in the row 540b) can be axially offset from the apices 544 of the cells 548 by an offset distance k. The valvular structure 504 is attached to and extends between the inflow end 508 (defined by apices 562 of the frame 402) and the outflow portions 554 of the commissure-supporting cells 522, which can have a distance l therebetween. In some examples, the distance l can generally correspond to a height of the valvular structure 504. As the commissure-supporting cells 522 are axially offset from the outflow end of the frame, the commissures 516 and the outflow edge portions 514 of the leaflets 512 of the valvular structure 504 can also be axially offset from the outflow end 510 of the frame 502 and the prosthetic valve 500. In some examples, the height j is in a range of 15 mm to 32 mm. In some examples, the offset distance k is in a range of 3 mm to 15 mm (such as, for example, a range of 3 mm to 7 mm). In some examples, the distance l is in a range of 12 mm to 25 mm. In some examples, a ratio of a height of the frame (l) to the distance l can be in a range of 1.1 to 2.7. In some examples, a ratio of a height of the frame (l) to the offset distance (k) can be in a range of 2 to 10.7. In one specific example, the height j is 25 mm, the offset distance k is 3 mm, and the distance l is 22 mm.

In some examples, at least the outflow portion 554 of each of the commissure-supporting cells 522 can be disposed at an oblique angle relative to the longitudinal axis of the frame 502 and angled inward toward the longitudinal axis of the frame 502. In some examples, the outflow portion 554 of each of the commissure-supporting cells 522 can be angled inward toward the longitudinal axis of the frame. In some examples, the outflow portion 554 of each of the commissure-supporting cells 522 can be angled inward at an oblique angle in a range of 20° to 30° relative to the longitudinal axis of the frame. In some examples, the outflow portions of the of the commissure-supporting cells can be parallel to or generally parallel to the longitudinal axis of the frame or angled away from the longitudinal axis of the frame.

The prosthetic valve 500 including the frame 502 can have one or more of the advantages of the prosthetic valve 400 (discussed above).

### Delivery Techniques

The prosthetic valves disclosed herein can be implanted using the techniques described below or other techniques.

In some examples, for implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

In some examples, for implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

In some examples, for implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

In some examples, another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In one or more of the foregoing delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame comprising an inflow end and an outflow end arranged along a longitudinal axis of the frame, the frame comprising: a first, outflow row of discontinuous cells, wherein the outflow end of the frame is defined by outflow apices of the discontinuous cells in the outflow row, wherein each discontinuous cell in the outflow row is unattached to other discontinuous cells to define a plurality of gaps in the frame between adjacent discontinuous cells within the outflow row; and a second row of cells upstream of the outflow row of cells comprising pairs of enlarged cells, wherein each cell in the outflow row extends from a respective pair of enlarged cells in the second row, wherein the second row further comprises a plurality of commissure-supporting cells disposed circumferentially between respective pairs of enlarged cells, wherein the enlarged cells are larger than the commissure-supporting cells and the discontinuous cells; and a valvular structure comprising a plurality of leaflets, the plurality of leaflets forming a plurality of commissures that are connected to struts of the commissure-supporting cells.

Example 2. The prosthetic heart valve of any example disclosed herein, particularly example 1, wherein each of the commissure-supporting cells is aligned with one of the gaps in the frame.

Example 3. The prosthetic heart valve of any example disclosed herein, particularly either of example 1 or example 2, wherein at least an outflow portion of each of the commissure-supporting cells is disposed at an oblique angle relative to the longitudinal axis of the frame, and is angled toward the longitudinal axis of the frame.

Example 4. The prosthetic heart valve of any example disclosed herein, particularly example 3, wherein at least the outflow portion of each of the commissure-supporting cells is disposed at an angle in a range of 5° to 35° relative to the longitudinal axis of the frame.

Example 5. The prosthetic heart valve of any example disclosed herein, particularly either of example 3 or example 4, wherein at least the outflow portion of each of the commissure-supporting cells is disposed at an angle in a range of 5° to 25° relative to the longitudinal axis of the frame.

Example 6. The prosthetic heart valve of any example disclosed herein, particularly any of examples 1-5, wherein at least an outflow portion of each discontinuous cell in the outflow row is angled inward toward the longitudinal axis of the frame.

Example 7. The prosthetic heart valve of any example disclosed herein, particularly any of examples 1-5, wherein at least an outflow portion of each discontinuous cell in the outflow row is angled outward away from the longitudinal axis of the frame.

Example 8. The prosthetic heart valve of any example disclosed herein, particularly any of examples 1-7, wherein the frame further comprises a third, inflow row of cells, wherein inflow apices of cells in the inflow row define the inflow end of the frame.

Example 9. The prosthetic heart valve of any example disclosed herein, particularly example 8, further comprising a fourth row of cells downstream of the inflow row of cells, each cell in the fourth row disposed circumferentially between the enlarged cells in each of the respective pairs of enlarged cells.

Example 10. The prosthetic heart valve of any example disclosed herein, particularly either of example 8 or example 9, wherein a ratio of a number of inflow apices to a number of outflow apices is four to one.

Example 11. The prosthetic heart valve of any example disclosed herein, particularly example 10, wherein the frame includes twenty four inflow apices.

Example 12. The prosthetic heart valve of any example disclosed herein, particularly either of example 8 or example 9, wherein a ratio of a number of inflow apices to a number of outflow apices is three to one.

Example 13. The prosthetic heart valve of any example disclosed herein, particularly example 12, wherein the frame includes eighteen inflow apices.

Example 14. The prosthetic heart valve of any example disclosed herein, particularly any of examples 1-13, wherein the frame includes six outflow apices.

Example 15. The prosthetic heart valve of any example disclosed herein, particularly any of examples 1-14, wherein an offset distance between apices of the commissure-supporting cells and the outflow apices of the discontinuous cells is in a range of 3 mm to 15 mm.

Example 16. The prosthetic heart valve of any example disclosed herein, particularly any of examples 1-15, wherein a ratio of a total height of the frame from the inflow end to the outflow end to a distance between the inflow end of the frame and apices of the commissure-supporting cells in a range of 1.1 to 3.2.

Example 17. An annular frame for a prosthetic heart valve, the frame comprising: an inflow end and an outflow end arranged along a longitudinal axis of the frame; a first, outflow row of discontinuous cells, wherein the outflow end of the frame is formed by outflow apices of the discontinuous cells in the outflow row, wherein each discontinuous cell in the outflow row is unattached to other discontinuous cells to define a plurality of gaps in the frame between adjacent discontinuous cells within the outflow row; and a second row of cells upstream of the outflow row of cells comprising pairs of enlarged cells, wherein each discontinuous cell in the outflow row extends from a respective pair of enlarged cells in the second row, wherein the second row further comprises a plurality of commissure-supporting cells disposed circumferentially between adjacent ones of the pairs of enlarged cells, wherein the enlarged cells are larger than the commissure-supporting cells and the discontinuous cells; wherein apices of the commissure-supporting cells are axially offset from the outflow end of the frame, toward the inflow end of the frame, by an offset distance.

Example 18. The annular frame of any example disclosed herein, particularly example 17, wherein at least an outflow portion of each of the commissure-supporting cells is angled inward toward the longitudinal axis of the frame and is disposed at an angle in a range of 20° to 30° relative to the longitudinal axis of the frame.

Example 19. The annular frame of any example disclosed herein, particularly either of example 17 or example 18, wherein at least an outflow portion of each of the discontinuous cells is angled inward toward the longitudinal axis of the frame and is disposed at an angle in a range of 1° to 15° relative to the longitudinal axis of the frame.

Example 20. The annular frame of any example disclosed herein, particularly any of examples 17-19, wherein each of the commissure-supporting cells is aligned with one of the gaps in the frame.

Example 21. The annular frame of any example disclosed herein, particularly any of examples 17-20, wherein the offset distance is in a range of 3 mm to 15 mm.

Example 22. A prosthetic heart valve comprising: an annular frame comprising an inflow end and an outflow end arranged along a longitudinal axis of the frame, the frame comprising: a plurality of pairs of cells circumferentially arranged in an outflow end portion of the frame, wherein a plurality gaps in the frame are defined at least in part by outflow portions of the pairs of cells, and wherein each of the gaps is disposed between respective adjacent ones of the pairs of cells; and a plurality of commissure-supporting cells, each of which is disposed circumferentially between a first pair of cells in the plurality of pairs of cells and an adjacent second pair of cells in the plurality of pairs of cells, wherein each of the commissure-supporting cells is aligned with one of the gaps in the frame and is axially offset from the outflow end of the frame toward the inflow end of the frame; and a valvular structure comprising a plurality of leaflets, the plurality of leaflets forming a plurality of commissures that are connected to struts of the commissure-supporting cells.

Example 23. The prosthetic heart valve of any example disclosed herein, particularly example 22, wherein at least an outflow portion of each of the commissure-supporting cells is disposed at an oblique angle relative to the longitudinal axis of the frame, and is angled toward the longitudinal axis of the frame.

Example 24. The prosthetic heart valve of any example disclosed herein, particularly example 23, wherein at least the outflow portion of each of the commissure-supporting cells is disposed at an angle in a range of 5° to 25° relative to the longitudinal axis of the frame.

Example 25. The prosthetic heart valve of any example disclosed herein, particularly any of example 22-24, wherein each of the pairs of cells includes enlarged cells, and wherein each of the enlarged cells is larger than one of the commissure-supporting cells.

Example 26. The prosthetic heart valve of any example disclosed herein, particularly any of examples 22-25, wherein cells in each of the pairs of cells are mirrored, and wherein the cells in each of the pairs of cells are non-overlapping with respect to each other when superimposed.

Example 27. The prosthetic heart valve of any example disclosed herein, particularly any of examples 22-25, wherein cells in each of the pairs of cells are mirrored, and wherein the cells in each of the pairs of cells are overlapping with respect to each other when superimposed.

Example 28. The prosthetic heart valve of any example disclosed herein, particularly any of examples 22-27, wherein an offset distance between apices of the commissure-supporting cells and the outflow end of the frame is in a range of 3 mm to 15 mm.

Example 29. The prosthetic heart valve of any example disclosed herein, particularly any of examples 22-28, wherein apices of cells of the pairs of cells define the outflow end of the frame.

Example 30. The prosthetic heart valve of any example disclosed herein, particularly any of examples 22-28, wherein the frame further comprises a row of discontinuous cells, wherein each of the discontinuous cells extends from one of the pairs cells and is unattached to others of the discontinuous cells, and wherein apices of the discontinuous cells define the outflow end of the frame.

The features described herein with regard to any example can be combined with other features described in any one or more of the other examples, unless otherwise stated. For example, any one or more of the features of one prosthetic valve can be combined with any one or more features of another prosthetic valve. As another example, any one or more features of one frame can be combined with any one or more features of another frame.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame comprising an inflow end and an outflow end arranged along a longitudinal axis of the frame, the frame comprising:
a first, outflow row of discontinuous cells, wherein the outflow end of the frame is defined by outflow apices of the discontinuous cells in the outflow row, wherein each discontinuous cell in the outflow row is unattached to other discontinuous cells to define a plurality of gaps in the frame between adjacent discontinuous cells within the outflow row; and
a second row of cells upstream of the outflow row of cells comprising pairs of enlarged cells, wherein each cell in the outflow row extends from a respective pair of enlarged cells in the second row, wherein the second row further comprises a plurality of commissure-supporting cells disposed circumferentially between respective pairs of enlarged cells, wherein the enlarged cells are larger than the commissure-supporting cells and the discontinuous cells; and
a valvular structure comprising a plurality of leaflets, the plurality of leaflets forming a plurality of commissures that are connected to struts of the commissure-supporting cells.

2. The prosthetic heart valve of claim 1, wherein each of the commissure-supporting cells is aligned with one of the gaps in the frame.

3. The prosthetic heart valve of claim 1, wherein at least an outflow portion of each of the commissure-supporting cells is disposed at an oblique angle relative to the longitudinal axis of the frame, and is angled toward the longitudinal axis of the frame.

4. The prosthetic heart valve of claim 1, wherein at least an outflow portion of each discontinuous cell in the outflow row is angled inward toward the longitudinal axis of the frame.

5. The prosthetic heart valve of claim 1, wherein at least an outflow portion of each discontinuous cell in the outflow row is angled outward away from the longitudinal axis of the frame.

6. The prosthetic heart valve of claim 1, wherein the frame further comprises a third, inflow row of cells, wherein inflow apices of cells in the inflow row define the inflow end of the frame.

7. The prosthetic heart valve of claim 6, further comprising a fourth row of cells downstream of the inflow row of cells, each cell in the fourth row disposed circumferentially between the enlarged cells in each of the respective pairs of enlarged cells.

8. The prosthetic heart valve of claim 1, wherein an offset distance between apices of the commissure-supporting cells and the outflow apices of the discontinuous cells is in a range of 3 mm to 15 mm.

9. The prosthetic heart valve of claim 1, wherein a ratio of a total height of the frame from the inflow end to the outflow end to a distance between the inflow end of the frame and apices of the commissure-supporting cells in a range of 1.1 to 3.2.

10. An annular frame for a prosthetic heart valve, the frame comprising:
an inflow end and an outflow end arranged along a longitudinal axis of the frame;
a first, outflow row of discontinuous cells, wherein the outflow end of the frame is formed by outflow apices of the discontinuous cells in the outflow row, wherein each discontinuous cell in the outflow row is unattached to other discontinuous cells to define a plurality of gaps in the frame between adjacent discontinuous cells within the outflow row; and
a second row of cells upstream of the outflow row of cells comprising pairs of enlarged cells, wherein each discontinuous cell in the outflow row extends from a respective pair of enlarged cells in the second row, wherein the second row further comprises a plurality of commissure-supporting cells disposed circumferentially between adjacent ones of the pairs of enlarged cells, wherein the enlarged cells are larger than the commissure-supporting cells and the discontinuous cells;
wherein apices of the commissure-supporting cells are axially offset from the outflow end of the frame, toward the inflow end of the frame, by an offset distance.

11. The annular frame of claim 10, wherein at least an outflow portion of each of the commissure-supporting cells is angled inward toward the longitudinal axis of the frame and is disposed at an angle in a range of 5° to 25° relative to the longitudinal axis of the frame.

12. The annular frame of claim 10, wherein at least an outflow portion of each of the discontinuous cells is angled inward toward the longitudinal axis of the frame and is disposed at an angle in a range of 1° to 15° relative to the longitudinal axis of the frame.

13. The annular frame of claim 10, wherein each of the commissure-supporting cells is aligned with one of the gaps in the frame.

14. The annular frame of claim 10, wherein the offset distance is in a range of 3 mm to 15 mm.

15. A prosthetic heart valve comprising:
an annular frame comprising an inflow end and an outflow end arranged along a longitudinal axis of the frame, the frame comprising:
a plurality of pairs of cells circumferentially arranged in an outflow end portion of the frame, wherein a plurality gaps in the frame are defined at least in part by outflow portions of the pairs of cells, and wherein each of the gaps is disposed between respective adjacent ones of the pairs of cells; and
a plurality of commissure-supporting cells, each of which is disposed circumferentially between a first pair of cells in the plurality of pairs of cells and an adjacent second pair of cells in the plurality of pairs of cells, wherein each of the commissure-supporting cells is aligned with one of the gaps in the frame and is axially offset from the outflow end of the frame toward the inflow end of the frame; and
a valvular structure comprising a plurality of leaflets, the plurality of leaflets forming a plurality of commissures that are connected to struts of the commissure-supporting cells.

16. The prosthetic heart valve of claim 15, wherein each of the pairs of cells includes enlarged cells, and wherein each of the enlarged cells is larger than one of the commissure-supporting cells.

17. The prosthetic heart valve of claim 15, wherein cells in each of the pairs of cells are mirrored, and wherein the cells in each of the pairs of cells are non-overlapping with respect to each other when superimposed.

18. The prosthetic heart valve of claim 15, wherein cells in each of the pairs of cells are mirrored, and wherein the cells in each of the pairs of cells are overlapping with respect to each other when superimposed.

19. The prosthetic heart valve of claim 15, wherein apices of cells of the pairs of cells define the outflow end of the frame.

20. The prosthetic heart valve of claim 15, wherein the frame further comprises a row of discontinuous cells, wherein each of the discontinuous cells extends from one of the pairs cells and is unattached to others of the discontinuous cells, and wherein apices of the discontinuous cells define the outflow end of the frame.
